(19) United States
(12) Patent Application Publication
(10) Pub. No.: US 2008/0083920 A1
(43) Pub. Date: Apr. 10, 2008

(54) COMPOSITION FOR PREVENTING LEANING IN FORMATION OF CAPACITOR, AND METHOD FOR MANUFACTURING CAPACITOR USING THE SAME

(75) Inventor: Geun Su Lee, Yongin-si (KR)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Hynix Semiconductor Inc., Icheon-si (KR)

(21) Appl. No.: 11/672,686

(22) Filed: Feb. 8, 2007

(30) Foreign Application Priority Data
Oct. 4, 2006 (KR) 10-2006-0097493

(51) Int. Cl. H01L 51/00; C07D 251/30

(52) U.S. Cl. 257/40; 544/220; 257/E51.02

(57) ABSTRACT

A method for manufacturing a capacitor of a semiconductor device by using a composition to prevent leaning of a capacitor. The method includes forming a barrier film and a capacitor oxide film over a semiconductor substrate including an interlayer insulation film with contact plugs for storage nodes; etching the capacitor oxide film and the barrier film until the contact plugs are exposed to form trenches for capacitors; forming lower electrodes for the storage nodes in the trenches; coating the composition over the lower electrodes and baking the composition to form a polymer layer connecting the upper portions of the lower electrodes; performing the wet-dip out process on the resulting structure to remove the capacitor oxide film; and performing an O₂ dry etching process to remove the polymer layer.

### CROSS-REFERENCE TO RELATED APPLICATIONS

This application claims benefit under 35 U.S.C. § 119 from Korean Patent Application No. 2006-97493, filed on Oct. 4, 2006, the entire content of which is incorporated herein by reference.

### BACKGROUND OF THE INVENTION

The present invention relates to a method of manufacturing a capacitor of a semiconductor device, and more particularly, a new material composition for preventing the collapse (leaning phenomena) of a three dimensional capacitor of a semiconductor device.

As the demand for smaller semiconductor devices increases, various techniques for manufacturing a capacitor with a high capacitance have been suggested.

In the capacitor, a dielectric film is interposed between a lower electrode for a storage node and an upper electrode for a plate node. The capacitance of the capacitor is proportional to the surface area of the electrode and the dielectric constant of the dielectric film, and inversely proportional to the gap between the electrodes, namely, the thickness of the dielectric film. A method using a dielectric film with a large dielectric constant, a method for reducing a thickness of a dielectric film, a method for increasing the surface area of the lower electrode, and a method for reducing a gap between electrodes have been used to manufacture a capacitor with a high capacitance.

With the increase of an integration degree, the size of the semiconductor memory device has been gradually reduced. It is thus difficult to manufacture a capacitor with a sufficient capacitance. Accordingly, research has been steadily conducted to improve the structure of the storage node. Concave type and cylinder type capacitors with a three-dimensional structure have been developed as a solution. Recently, the cylinder type capacitor using both the internal area and the external area as the node area has been more popularly used than the concave type capacitor using the internal area as the node area.

### BRIEF SUMMARY OF THE INVENTION

Disclosed herein is a composition for efficiently preventing the collapse of the lower electrodes in formation of a cylinder type capacitor.

The composition comprises a compound represented by the following Chemical Formula 1 and an organic solvent.

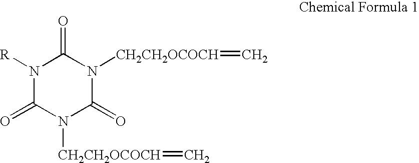

Wherein, R represents linear or side chain C₁˜C₁₀ alkyl, linear or side chain C₁˜C₁₀ alkyl substituted by hydroxyl group, C₁˜C₁₀ alkyl containing linear or side chain C₃˜C₁₀ ester group, or C₁˜C₁₀ alkyl containing linear or side chain C₂˜C₁₀ ether group.

A method for manufacturing a capacitor of a semiconductor device by using the composition for preventing leaning of the capacitor, comprising: sequentially forming a barrier film and a capacitor oxide film over a semiconductor substrate including an interlayer insulation film with contact plugs for storage nodes; sequentially etching the capacitor oxide film and the barrier film until the contact plug is exposed to form a trenches for capacitors; forming lower electrodes for storage nodes in the trenches; coating the disclosed composition over the lower electrodes and baking the disclosed composition to form a polymer layer connecting the upper portions of the lower electrodes; performing the wet-dip out process on the resulting structure to remove the capacitor oxide film; and performing an O₂ dry etching process to remove the polymer layer.

A method for manufacturing a capacitor which can improve reliability of a device and attain a sufficient capacitance in spite of a high integration tendency, by using the composition.

### BRIEF DESCRIPTION OF THE DRAWINGS

The embodiments of the present invention will become more apparent when described with reference to the accompanying drawings, in which:

FIGS. 1a and 1b are cross-sectional diagrams illustrating a method for manufacturing a cylinder type capacitor;

FIG. 2 is an SEM photograph showing collapse of a lower electrode during formation of the capacitor;

FIGS. 3a to 3f are cross-sectional diagrams illustrating a method for manufacturing a capacitor according to an embodiment of the present invention;

FIG. 4a is an SEM photograph showing a substrate with a polymer layer connecting upper portions of the lower electrodes in accordance with the present invention;

FIGS. 4b and 4c are SEM photographs showing the upper portions of the lower electrodes after the polymer layer is removed from the substrate of FIG. 4a by an O₂ etching process;

FIG. 5a is an SEM photograph showing the capacitors lower electrodes formed by Example 3; and

FIG. 5b is an SEM photograph showing the capacitors lower electrodes formed by Example 4.

### DESCRIPTION OF SPECIFIC EMBODIMENTS

There is provided a material composition for efficiently preventing the collapse of the lower electrodes during formation of a cylinder type capacitor. Also a method for manufacturing a capacitor of a semiconductor device.

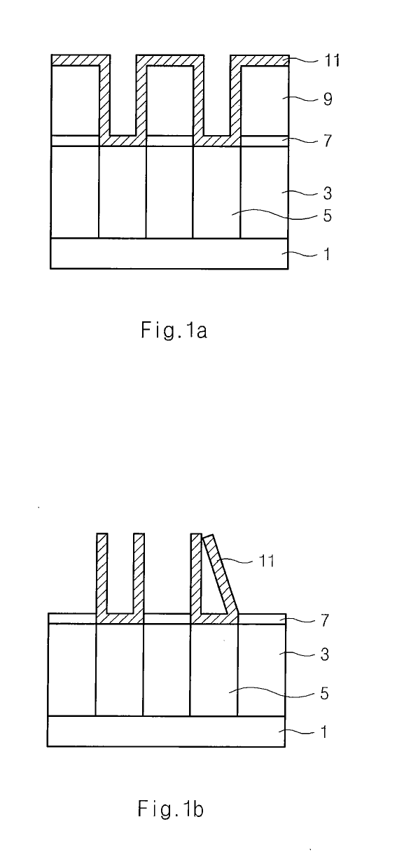

FIGS. 1a and 1b are cross-sectional diagrams illustrating a method for manufacturing a three-dimensional cylinder type capacitor.

FIG. 1a shows an interlayer insulation film 3 formed over a semiconductor substrate 1 on which semiconductor circuits such as transistors and bit lines have been formed. The Storage node contact holes exposing parts of the semiconductor substrate 1 are formed by etching the interlayer insulation film 3.

Storage node contact plugs 5 are filled into the storage node contact holes. A nitride film 7 which is an etching barrier film and a capacitor oxide film 9 which decides the height of lower electrodes are sequentially deposited over the interlayer insulation film 3 including the storage node contact plugs 5.

Trenches for storage nodes (not shown) are formed by dry-etching the capacitor oxide film 9. Lower electrodes for storage nodes 11 are formed in the trenches.

A wet etching process using a wet chemical and a rinsing process are sequentially carried out on the resulting structure of FIG. 1a. A wet-dip out process for drying is performed on the resulting structure to remove the capacitor oxide film 9.

On the other hand, in order to secure the capacitance of the capacitor, the method increases the surface area of the lower electrodes 11 by increasing the height of the capacitor oxide film 9.

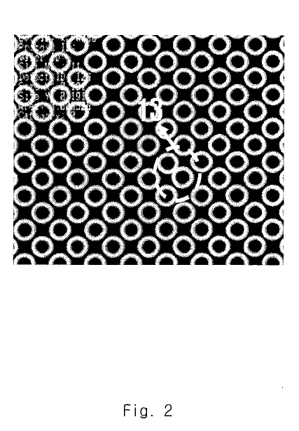

FIG. 2 shows a leaning phenomenon 13 where the lower electrodes are collapsed and are in contact with each other.

When the aspect ratio of the lower electrodes increases, the moisture that infiltrates between the lower electrodes 1 in the wet-dip out process for removing the capacitor oxide film 9 causes surface tension. This surface tension is generated between the adjacent lower electrodes 11, which pulls them together, thereby causing the lower electrodes 11 to collapse and contact each other during the drying process for removing moisture. The phenomenon is called leaning phenomenon.

If the line-width between the capacitors decreases from the size reduction of the device, the bottom line-width of the capacitor decreases, and/or the height of the capacitor increases, causing the leaning phenomenon 13 to increase.

In an desirable embodiment of the present invention, the disclosed composition for preventing the collapse of lower electrodes comprises a compound represented by following Chemical Formula 1 and an organic solvent.

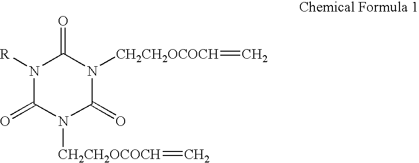

wherein, R represents linear or side chain C₁˜C₁₀ alkyl, linear or side chain C₁˜C₁₀ alkyl substituted by hydroxyl group, C₁˜C₁₀ alkyl containing linear or side chain C₃˜C₁₀ ester group, or C₁˜C₁₀ alkyl containing linear or side chain C₂˜C₁₀ ether group.

Preferably, R represents linear or side chain C₁˜C₅ alkyl, linear or side chain C₁˜C₅ alkyl substituted by hydroxyl group, C₁˜C₅ alkyl containing linear or side chain C₃˜C₆ ester group, or C₁˜C₅ alkyl containing linear or side chain C₂˜C₆ ether group. More preferably, R represents —CH₂CH₂OCOCH═CH₂ or —CH₂CH₂OH.

This compound has a molecular weight of 300 to 30000.

Preferably, the compound of Chemical Formula 1 is present in an amount ranging from 1 to 5 wt %, based on the total weight of the composition.

An organic solvent not specifically limited to preparing a photoresist composition can be used as the organic solvent. For example, C₁˜C₁₀ alkyl alcohol, C₂˜C₁₀ ether, C₃˜C₁₀ ketone compound or combinations thereof can be used as the organic solvent. Preferably, the organic solvent is selected from the group consisting of propylene glycol methyl ether acetate (PGMEA), propylene glycol monomethyl ether (PGME), ethyl lactate, cyclohexanone, γ-butyrolactone, n-butanol, 2-butanol, 1-pentanol, 2-pentanol and combinations thereof.

The amount of organic solvent ranges from 95 to 99 wt %, based on the total weight of the composition. It is impossible to obtain a polymer layer having a sufficient thickness when the organic solvent is present in the amount of more than 99 wt %, and it is difficult to uniformly fill the polymer layer in the trench when the organic solvent is present in an amount of less than 95 wt %.

In an embodiment of the present invention, a method for manufacturing a capacitor of a semiconductor device by using the composition for preventing collapse of the capacitor comprises of:
sequentially forming a barrier film and a capacitor oxide film over a semiconductor substrate including an interlayer insulation film with contact plugs for storage nodes;
sequentially etching the capacitor oxide film and the barrier film until the contact plug is exposed to form a trench for capacitors; forming lower electrodes for storage nodes in the trenches;
coating the disclosed composition over the lower electrodes and baking the disclosed composition to form a polymer layer connecting the upper portions of the lower electrodes;
performing the wet-dip out process on the resulting structure to remove the capacitor oxide film; and
performing an O₂ dry etching process to remove the polymer layer.

The method further comprises performing a partial etching process using O₂ gas after forming the polymer layer and before performing the wet-dip out process.

The method of the disclosed invention comprises forming the composition containing a compound known as a curing agent over the capacitor oxide film, and baking the resulting structure, thereby forming the cured polymer layer connecting the lower electrodes. The cured polymer layer has low solubility to a wet chemical used in the succeeding wet-dip out process. Therefore, when the capacitor oxide film is removed, the polymer layer is not removed. The polymer layer serves as a support to prevent the collapse of the lower electrodes during the wet-dip out process. As a result, the leaning phenomenon of the adjacent lower electrodes does not occur.

A composition for preventing the collapse of the capacitor and a method for manufacturing a capacitor using the same in accordance with embodiments of the disclosed invention will now be described in detail with reference to the accompanying drawings. As the present invention may be embodied in various forms, it should be understood that the scope of the present invention is not limited by any of the details of the following embodiments. The embodiments of the present invention are provided to efficiently describe the present invention to those skilled in this field. Therefore, shapes and sizes of elements may not be drawn to scale in the figures for more accurate explanation. In addition, the same elements are denoted by the same reference numerals in different drawings.

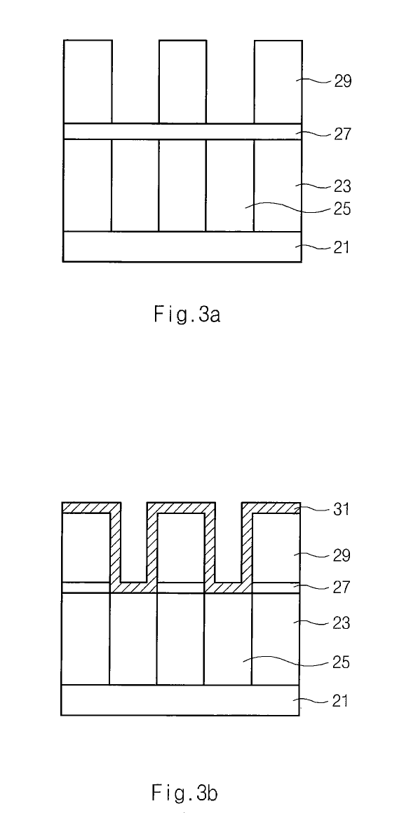

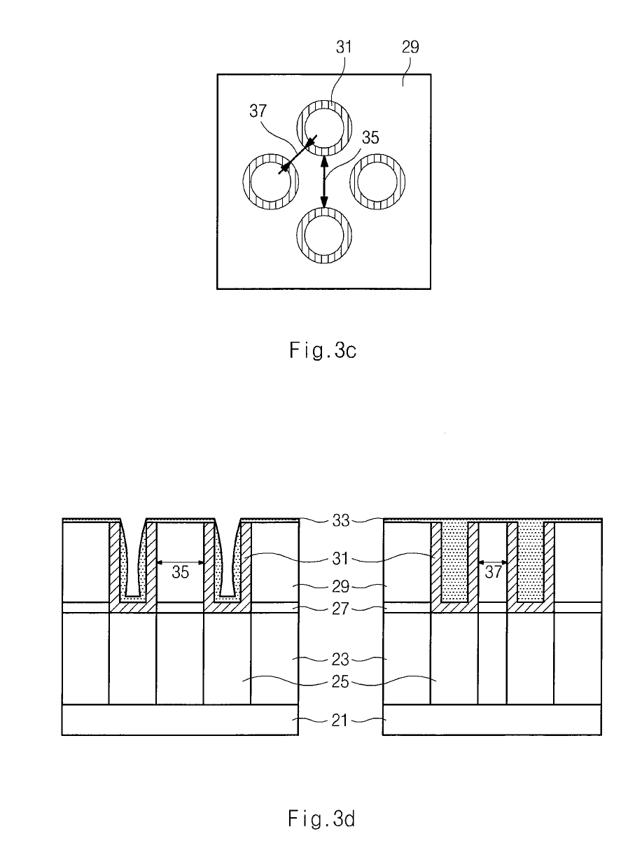

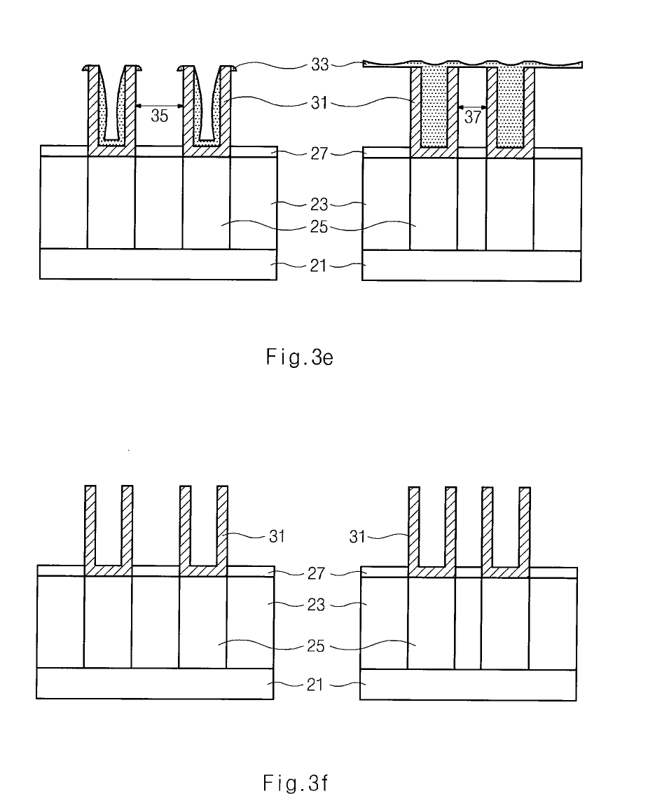

FIGS. 3a to 3f are cross-sectional diagrams illustrating a method for manufacturing a three-dimensional cylinder type capacitor according to an embodiment of the present invention

FIG. 3a shows an interlayer insulation film 23 formed over a semiconductor substrate 21 on which semiconductor circuits such as transistors (not shown) and bit lines (not shown) have been formed. Storage node contact holes (not shown) exposing parts of the semiconductor substrate 21 are formed by etching the interlayer insulation film 23.

Storage node contact plugs 25 are filled into the storage node contact holes. A nitride film 27 which is an etching barrier film and a capacitor oxide film 29 which decides the height of the lower electrodes are sequentially deposited over the interlayer insulation film 21 including the storage node contact plugs 25.

The capacitor oxide film 29 is formed of phosphosilicate glass (PSG) or plasma enhanced tetra-ethyl-ortho-silicate (PE-TEOS).

Trenches (not shown) for capacitors exposing the storage node contact plugs 25 is formed by sequentially etching the capacitor oxide film 29 and the barrier film 27.

FIG. 3b shows a lower electrode 31 layer for storage nodes formed in the trenches of FIG. 3a.

FIG. 3c shows the separated lower electrode 31 obtained by performing a CMP process until the capacitor oxide film 29 is exposed. The wide line-width 35 between the lower electrodes 31 is about 103 nm, and the narrow line-width 37 between the lower electrodes 31 is about 40 nm.

FIG. 3d shows a polymer layer 33 formed by coating the composition for preventing leaning on the whole surface of the resulting structure, and baking the resulting structure.

The polymer layer 33 is formed over the capacitor oxide film 29 at a thickness of about 10 to 150 nm, preferably, 10 to 30 nm. If the thickness of the polymer layer 33 is over 150 nm, it takes a long time to carry out a subsequent partial etching process. Therefore the process cannot be stably performed. If the thickness of the polymer layer 33 is below 10 nm, the polymer layer 33 is removed in the partial etching process or a wet-dip out process, thereby causing the leaning phenomenon and collapsing the lower electrodes.

The baking process is performed at over 200° C. for 40 to 90 seconds.

On the other hand, when the polymer layer 33 is formed, in the narrow line width 37 between the lower electrodes 31, the polymer layer 33 is filled in the whole inner portions of the lower electrodes 31. However, in the wide line width 35 between the lower electrodes 31, the polymer layer 33 is formed on the inner sidewalls of the lower electrodes 31.

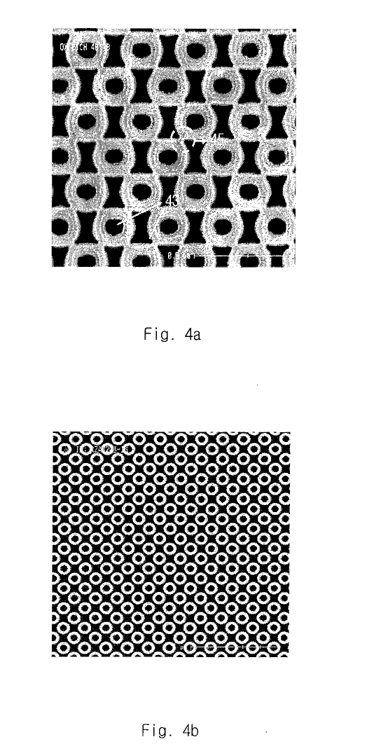

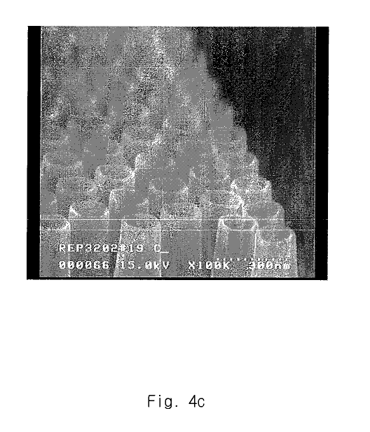

FIG. 4a shows that the partial etching process using O₂ etching gas is performed on the resulting structure of FIG. 3d until the capacitor oxide film 29 is exposed.

As a result, the polymer layer 33 existing in the wide line-width 35 between the lower electrodes 31 and the polymer layer 33 in the lower electrodes 31 is partially removed 43. However, the polymer layer 33 existing in the narrow line-width 37 between the lower electrodes 31 is not removed but remains 45 to connect the upper portions of the lower electrodes 31.

The partial etching process is performed for 30 seconds to 1 minute.

The polymer layer 33 can be formed of an amorphous carbon, instead of using the composition of the present invention. However, since the equipment for coating the polymer layer 33 has higher productivity than the equipment for depositing amorphous carbon, the process cost can be cut down by using the composition of the present invention.

FIG. 3e shows that the wet-dip out process using a wet chemical is performed on the resulting structure after the partial etching process, thereby removing the capacitor oxide film 29.

Here, the polymer layer 33 is not removed by the wet-dip out process. In addition, the capacitor oxide film 29 formed under the lower part of the polymer layer of the narrow line-width 37 is not removed due to the polymer layer 33 remaining on the narrow line-width 37.

The wet-dip out process is performed by using buffered oxide etchant (BOE) solution such as 5% HF aqueous solution (HF:DI=1:20). Preferably, the wet-dip out process comprises; performing a wet etching process where the substrate is dipped in the BOE aqueous solution for 20 minutes to 1 hour, a rinsing with distilled water and alcohol, and drying the substrate to remove the moisture.

FIGS. 4b and 4c show that a dry etching process using O₂ gas is performed on the resulting structure of FIG. 3e, thereby removing the polymer layer 33 and the capacitor oxide film 29.

The dry etching process is carried out for 30 seconds to 1 minute.

FIG. 3f shows the vertical lower electrodes 31 for the storage nodes are formed without collapse.

In the conventional method for forming the cylinder type capacitor, the wet-dip out process is performed without coating the polymer layer on the capacitor oxide film and the lower electrodes. Accordingly, the lower electrode are collapsed and come into contact with each other in the narrow line width regions due to the surface tension of the wet etching solution.

However, in accordance with the disclosed invention, the polymer layer formed by the simple process serves as a support for connecting the upper portions of the lower electrodes. Thus, in the subsequent process, the surface tension is not generated between the lower electrodes in the narrow line width regions. Moreover, the polymer layer can be easily removed by the dry etching process using the gas which does not have the surface tension. In the process for manufacturing the capacitor using the method of the disclosed invention, the leaning phenomenon of the lower electrodes does not occur. As a result, the method of the disclosed invention stabilizes the semiconductor process, and obtains a capacitor with sufficient capacitance.

In addition, the present invention also provides a semiconductor device manufactured by the method for manufacturing the semiconductor device including the pattern formation process.

The present invention will now be explained in detail by the following examples, which are not intended to be limiting.

I. Preparation of Composition of Disclosed Invention

### EXAMPLE 1

1 g of the compound of Chemical Formula 1 (R is CH₂CH₂OCOCH═CH₂) (M-315, produced by TOAGOSE) was dissolved in 40 ml of PGMEA.

### EXAMPLE 2

1 g of the compound of Chemical Formula 1 (R is CH₂CH₂OH) (M−215, produced by TOAGOSE) was dissolved in 40 ml of PGMEA.

II. Method for Forming Capacitor of Disclosed Invention

### EXAMPLE 3

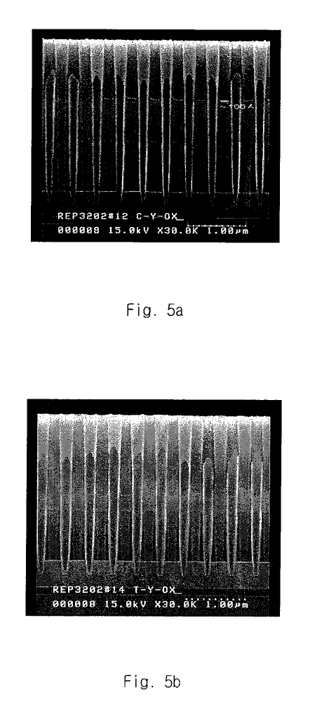

The composition of Example 1 is coated over a semiconductor substrate on which a capacitor oxide film and lower electrodes has been formed at 200 rpm. The composition of Example 1 is baked to form a polymer layer having a thickness of 20 nm at 200° C. for 60 seconds. The polymer layer was partially etched for 50 seconds by using O₂ gas. After the etching process, the semiconductor substrate is dipped in 5% HF aqueous solution for 35 minutes to remove the capacitor oxide film, and rinsed by using distilled water. Thereafter, the polymer layer was removed by carrying out an etching process using O₂ gas for 50 seconds (refer to FIG. 5a).

### EXAMPLE 4

The composition of Example 2 is coated over a semiconductor substrate on which a capacitor oxide film and lower electrodes had been formed at 200 rpm. The composition of Example 1 is baked to form a polymer layer having a thickness of 20 nm at 200° C. for 60 seconds. The polymer layer was partially etched for 50 seconds by using O₂ gas. After the etching process, the semiconductor substrate is dipped in 5% HF aqueous solution for 35 minutes to remove the capacitor oxide film, and rinsed by using distilled water. Thereafter, the polymer layer was removed by carrying out an etching process using O₂ gas for 50 seconds (refer to FIG. 5b).

As discussed earlier, the method of the disclosed invention prevents collapse of the capacitor in the formation of the capacitor, thereby securing the sufficient height of the storage node of the capacitor. As a result, the capacitor can be manufactured with a high capacitance.

The foregoing embodiment and advantages are merely examples and are not limiting. The present teaching can be readily applied to other types of apparatuses. Also, the description of the embodiments of the present invention is intended to be illustrative, and not to limit the scope of the claims, and many alternatives, modifications, and variations will be apparent to those skilled in the art.

What is claimed is:

1. A composition for preventing leaning of a capacitor comprising:
a compound represented by following Chemical Formula 1 and an organic solvent, 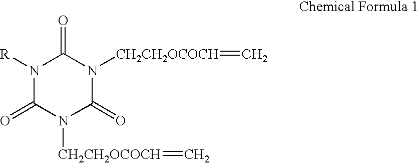
wherein, R is selected from the group consisting of a linear or side chain C₁˜C₁₀ alkyl; a linear or side chain C₁˜C₁₀ alkyl substituted by hydroxyl group; a C₁˜C₁₀ alkyl containing linear or side chain C₃˜C₁₀ ester group; and a C₁˜C₁₀ alkyl containing linear or side chain C₂˜C₁₀ ether group.

2. The composition according to claim 1, wherein R is selected from the group consisting of a linear or side chain C₁˜C₅ alkyl; a linear or side chain C₁˜C₅ alkyl substituted by hydroxyl group, a C₁˜C₅ alkyl containing linear or side chain C₃˜C₆ ester group; and a C₁˜C₅ alkyl containing linear or side chain C₂˜C₆ ether group.

3. The composition according to claim 1, wherein R represents —CH₂CH₂OCOCH═CH₂ or —CH₂CH₂OH.

4. The composition according to claim 1, wherein the compound of Chemical Formula 1 has a molecular weight ranging from 300 to 30,000.

5. The composition according to claim 1, wherein the compound of Chemical Formula 1 is present in an amount ranging from 1 to 5 wt %, based on the total weight of the composition.

6. The composition according to claim 1, wherein the organic solvent is selected from the group consisting of C₁˜C₁₀ alkyl alcohol, C₂-C₁₀ ether, C₃˜C₁₀ ketone compound and combinations thereof.

7. The composition according to claim 6, wherein the organic solvent is selected from the group consisting of propylene glycol methyl ether acetate, propylene glycol monomethyl ether, ethyl lactate, cyclohexanone, γ-butyrolactone, n-butanol, 2-butanol, 1-pentanol, 2-pentanol and combinations thereof.

8. The composition according to claim 1, wherein the organic solvent is present in an amount ranging from 95 to 99 wt %, based on the total weight of the composition.

9. A method for manufacturing a capacitor, the method comprising:
forming a barrier film and a capacitor oxide film over a semiconductor substrate including an interlayer insulation film with contact plugs for storage node;
etching the capacitor oxide film and the barrier film until the contact plugs are exposed to form trenches for capacitors;
forming lower electrodes for the storage nodes in the trenches;
coating a composition over the lower electrode, and baking the composition to form a polymer layer connecting upper portions of the lower electrodes;
removing the capacitor oxide film from the resulting structure; and
removing the polymer layer by performing an O₂ dry etching process,
wherein the composition comprises:
a compound represented by following Chemical Formula 1 and an organic solvent, 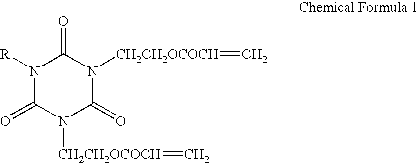
wherein, R is selected from the group consisting of a linear or side chain C₁˜C₁₀ alkyl; a linear or side chain C₁˜C₁₀ alkyl substituted by hydroxyl group; a C₁˜C₁₀ alkyl containing linear or side chain C₃˜C₁₀ ester group; and a C₁˜C₁₀ alkyl containing linear or side chain C₂˜C₁₀ ether group.

10. The method according to claim 9, further comprising performing a partial etching process using O₂ gas after forming the polymer layer and before removing the capacitor oxide film.

11. The method according to claim 9, wherein the capacitor oxide film includes phosphosilicate glass or plasma enhanced tetra-ethyl-ortho-silicate, wherein the capacitor oxide film is removed using a wet-dip process.

12. The method according to claim 9, wherein the polymer layer is formed at a thickness ranging from 10 to 150 nm.

13. The method according to claim 12, wherein the polymer layer is formed at a thickness ranging from 10 to 30 nm.

14. The method according to claim 11, wherein removing the capacitor oxide film comprises:
wet-etching the substrate using the buffered oxide etchant solution;
rinsing the substrate using the distilled water or alcohol; and
drying the substrate.

15. The method according to claim 14, wherein removing the capacitor oxide film involves dipping the substrate in the 5% HF aqueous solution for 20 minutes to 1 hour.
